(19) United States
(12) Patent Application Publication
(10) Pub. No.: US 2013/0203058 A1
(43) Pub. Date: Aug. 8, 2013

(54) COMPOSITE ASSAY FOR DETECTING A CLINICAL CONDITION

(71) Applicant: Anthony P. Shuber, Mendon, MA (US)

(72) Inventor: Anthony P. Shuber, Mendon, MA (US)

(21) Appl. No.: 13/757,287

(22) Filed: Feb. 1, 2013

Related U.S. Application Data
(60) Provisional application No. 61/594,102, filed on Feb. 2, 2012.

(51) Int. Cl. C12Q 1/68

(52) U.S. Cl. CPC C12Q 1/6886; C12Q 1/6883
USPC 435/6.11

(57) ABSTRACT

The invention generally relates to methods for screening patients for one or more clinical conditions using a composite assay. According to certain aspects, methods of the invention involve isolating at least one nucleic acid from a biological sample obtained from the subject, detecting at least one sequence mutation and a chromosomal abnormality in the at least one nucleic acid in a single assay format, and identifying a clinical condition in said subject when both the sequence mutation and the chromosomal abnormality are present.

### RELATED APPLICATION

This application claims the benefit of and priority to provisional U.S. patent application Ser. No. 61/594,102, filed on Feb. 2, 2012, the entirety of which is incorporated herein by reference.

### FIELD OF THE INVENTION

The invention generally relates to methods for screening patients for one or more clinical conditions using a composite assay.

### BACKGROUND

Clinical screening is desirable to detect a disease or other clinical condition, such as cancer, inflammation, or autoimmune disease, prior to the presentation of related symptoms. Such early stage detection allows for treatment of the disease or condition when treatment is more effective and less costly. However, early screening may lead to false positives or false negatives. These incorrect diagnoses can cause undue stress on the patient in the form of anxiety, physical discomfort or side effects from a medical treatment that was not needed. Additionally, incorrect diagnoses may result in the unnecessary use of other medical resources, or a loss in confidence by the patient for the efficacy of a needed medical test. In worst case situations, an incorrect diagnosis can delay necessary treatment for a medical condition and result in a terminal illness.

Diagnostic assays based upon the measurement of multiple biomarkers have been used as a way to increase the accuracy of a diagnostic screening test. For example, assays have been proposed in which the gene expression of several genes is measured in order to assess clinical status. Multiple protein analytes have also been used to screen for the presence of any of multiple disorders where the diagnosis is unclear. Oftentimes however, increasing the number of biomarkers measured in a given screening assay does not provide a significant improvement over the measurement of a single biomarker.

### SUMMARY OF THE INVENTION

The invention provides methods for assessing clinical condition by taking into account different types of underlying genetic information as well as gene expression data. Methods of the invention result in improved ability to diagnose the presence of a clinical condition or disorder. Methods of the invention recognize that a single genetic marker type is insufficient to diagnose and characterize a clinical condition with high sensitivity and specificity. According to the invention, methods that comprise multimodal analysis have greater sensitivity and specificity in the diagnosis and characterization of disease.

In one embodiment of the invention, the methods of the invention involve isolating at least one nucleic acid from a biological sample, using a single assay format to detect a sequence mutation and a chromosomal abnormality in the at least one nucleic acid, and identifying a clinical condition in the patient when both a sequence mutation and chromosomal abnormality are present. The isolated nucleic acid is preferably DNA (e.g., genomic DNA). The detected sequence mutation and chromosomal abnormality can occur on the same chromosome or on different chromosomes.

The biological sample can be any bodily fluid such as blood, a blood fraction, saliva, sputum, urine, semen, transvaginal fluid, cerebrospinal fluid, or stool, or a cell or tissue sample from any bodily organ such as the brain, mouth, throat, esophagus, stomach, lymph node, stomach, colon (large or small), kidney, bladder, liver, pancreas, skin, muscle, bone, bone marrow, ovary, vagina, cervix, uterus, testicle or prostate.

Sequence mutations contemplated by the present invention include, point mutations such as deletions, insertions, transitions, transversions, frameshift mutations, nonsense mutations, missense mutations, single nucleotide polymorphisms. Methods of the invention additionally contemplate the presence of viral DNA insertions in patient sequences. Partial or whole chromosomal abnormalities include but are not limited to, deletions, microdeletions, translocations, inversions, duplications, ring chromosomes, isochrome formation, chromosome copy gain, chromosome copy loss, and loss of heterozygosity (LOH).

Screening for other types of genetic markers are contemplated by the methods of the invention and include but are not limited to the detection of chemical sequence modifications. Chemical sequence modifications include, but are not limited to, acetylation, glycosylation, phosphorylation, and methylation. In a particular embodiment, the methods of the invention include screening for the presence or absence of methylation of a nucleic acid sequence, such as de-methylation, methylation, hypomethylation and hypermethylation.

The methods of the invention are suitable for the detection of any clinical condition or disorder. In a particular embodiment, the methods of the invention are contemplated for use in the clinical detection of cancer, such as breast cancer, ovarian cancer, uterine cancer, cervical cancer, ovarian cancer, prostate cancer testicular cancer, lung cancer, stomach cancer, brain cancer, colon cancer, kidney cancer, pancreatic cancer, skin cancer and bladder cancer.

In a particular embodiment, the invention provides methods for detecting cancer via the detection of a sequence mutation and loss of heterozygosity for at least two distinct genes. The two distinct genes may occur at different locations on the same chromosome. Alternatively, the two distinct genes may occur on different chromosomes. In a particular embodiment, the at least two distinct genes include FGFR3 and p53, where a sequence mutation in FGFR3 is detected and LOH in p53 is detected. Hypermethylation of one or more of the same or different gene sequences can optionally be detected in combination with the gene sequence mutation and LOH.

The methods of the invention are preferably performed in a single assay format. In one embodiment, the screening assay is a sequencing assay. Suitable sequencing methods include, but are not limited to, single molecule sequencing techniques. Alternatively, the methods of the invention can be performed using one or more methods such as real-time or quantitative PCR, digital PCR, and/or PCR in flowing or stationary droplets, well plates, slugs or fluid flowing segments, and the like, microarrays for subsequent fluorescent or non-fluorescent detection, barcode mass detection using a mass spectrometric methods, detection of emitted radiowaves, detection of scattered light from aligned barcodes, fluorescence detection using quantitative PCR or digital PCR methods, Northern blot, selective hybridization, cleaved amplified polymorphic sequence analysis, short tandem repeat analysis, the use of supports coated with oligonucleotide probes, amplification of the nucleic acid by RT-PCR, quantitative PCR or ligation-PCR, etc.

These and other aspects of the invention are described in further detail in the description and claims that follow.

### DETAILED DESCRIPTION OF THE INVENTION

Methods of the invention provide a sensitive and specific test for detecting and diagnosing different diseases or disorders, particularly cancer. The invention recognizes that a single type of genetic information may be insufficient for diagnosis and classification of a disease or disorder. Rather, the assessment of a combination of different types of genetic markers, provides a much more robust analysis tool.

Methods of the invention rely on the detection of different types of genetic markers in order to achieve superior diagnostic accuracy. The different types of genetic markers measured may occur on the same chromosome, or on different chromosomes. Preferably, the detection of the different types of genetic markers is achieved in a single assay format.

In certain aspects, both a sequence mutation and a chromosomal abnormality is detected from a patient sample. The detection of a sequence mutation alone may not be predictive because single biomarkers oftentimes have a high false positive or false negative rate. In combination with the detection of chromosomal abnormality, the desired predictive values are achieved. The sequence mutation and the chromosomal abnormality may occur on the same chromosome, or on different chromosomes. Optionally, one or more types of chemical sequence modifications (e.g., hypermethylation) may be detected in combination with a sequence mutation and chromosomal abnormality to further improve diagnostic accuracy.

Accordingly, methods of the invention provide for a evaluating a patient sample for any combination of two or more characteristics in order to form a more complete diagnostic profile for a clinical condition or disorder.

### Obtaining a Biological Sample

Methods of the invention involve obtaining a biological sample, from a subject. Samples may include any bodily fluid such as blood, a blood fraction, saliva, sputum, urine, semen, transvaginal fluid, cerebrospinal fluid, or stool. Other such samples may include one or more cells or a tissue biopsy, such as a cell or biopsy from the brain, mouth, throat, esophagus, stomach, lymph node, stomach, intestine (large or small), kidney, bladder, liver, pancreas, skin, muscle, bone, bone marrow, breast, ovary, vagina, cervix, uterus, testicle or prostate.

The sample may be obtained by methods known in the art, such as a phlebotomy, cheek swab, fine needle aspiration, core needle biopsy, vacuum assisted biopsy, direct and frontal lobe biopsy, shave biopsy, punch biopsy, excisional biopsy, or cutterage biopsy. Once the sample is obtained, nucleic acids are extracted to assess nucleic acid sequence mutations, chemical sequence modifications, and/or chromosomal abnormalities.

### Nucleic Acids

Nucleic acids may be obtained by methods known in the art. Generally, nucleic acids can be extracted from a biological sample by a variety of techniques such as those described by Maniatis, et al., Molecular Cloning: A Laboratory Manual, Cold Spring Harbor, N.Y., pp. 280-281, (1982), the contents of which is incorporated by reference herein in its entirety. The isolated nucleic acid molecules may be single-stranded, double-stranded, or double-stranded with single-stranded regions (for example, stem- and loop-structures). The isolated nucleic acid can be deoxyribonucleic acid (DNA) and/or ribonucleic acid (RNA). In a particular embodiment, genomic DNA is isolated from the biological sample.

It may be necessary to first prepare an extract of the cell and then perform further steps—i.e., differential precipitation, column chromatography, extraction with organic solvents and the like—in order to obtain a sufficiently pure preparation of nucleic acid. Extracts may be prepared using standard techniques in the art, for example, by chemical or mechanical lysis of the cell. Extracts then may be further treated, for example, by filtration and/or centrifugation and/or with chaotropic salts such as guanidinium isothiocyanate or urea or with organic solvents such as phenol and/or HCCl3 to denature any contaminating and potentially interfering proteins.

### Genetic Markers

Methods of the invention involve the detection of at least two types of genetic markers from the patient's isolated nucleic acid. The different genetic markers preferably include a sequence mutation in one or more genes, and partial or whole chromosomal abnormalities.

Sequence mutations contemplated by the present invention include, point mutations such as deletions, insertions, transitions, transversions, frameshift mutations, nonsense mutations, missense mutations, single nucleotide polymorphisms. Methods of the invention additionally contemplate the presence of viral DNA insertions in patient sequences. In certain embodiments the methods of the invention are used to identify nucleic acid sequence alterations that are causally implicated in cancer.

Chromosomal abnormalities contemplated by the present invention include but are not limited to, deletions, microdeletions, translocations, inversions, duplications, aneuploidy, ring chromosomes, isochrome formation, chromosome copy gain, chromosome copy loss, loss of heterozygosity (LOH) and Loss of Imprinting (LOI).

LOH is a common occurrence in patients with cancer, and indicates the absence of a functional tumor suppressor gene in the lost region. Many people with LOH remain healthy because there still is one functional gene left on the other chromosome of the chromosome pair. However, the remaining copy of the tumor suppressor gene can be inactivated by a point mutation, leaving no tumor suppressor gene to protect the body and result in the LOH phenotype.

LOI is a result of the loss of the normal function of an imprinted gene. Genomic imprinting is a genetic phenomenon wherein genes are expressed in a parent-of-origin-specific manner. Imprinted alleles are silenced such that the genes are either expressed only from the non-imprinted allele inherited from the mother (e.g. H19 or CDKN1C), or in other instances from the non-imprinted allele inherited from the father (e.g. IGF-2). Genomic imprinting is an epigenetic process that involves methylation and histone modifications in order to achieve monoallelic gene expression without altering the genetic sequence. These epigenetic marks are established in the germline and are maintained throughout all somatic cells of an organism. Appropriate expression of imprinted genes is important for normal development, with numerous genetic diseases associated with imprinting defects including Beckwith-Wiedemann syndrome, Silver-Russell syndrome, Angelman syndrome and Prader-Willi syndrome. LOI has been implicated causally in various cancers, including, but not limited to, breast and ovarian cancers. LOI can be detected through the sequencing as described in S.R. Bischoff, et al (Biol Reprod. 2009 November; 81(5): 906-920).

Screening for additional types of genetic markers may optionally be combined with the detection of one or more sequence mutations and chromosomal abnormalities. In certain aspects, the methods of the invention include the detection of a sequence mutation, a chromosomal abnormality and a chemical sequence modification. Chemical sequence modifications include, but are not limited to, acetylation, glycosylation, phosphorylation, and methylation.

In a particular embodiment, the methods of the invention optionally include screening for the presence or absence of methylation of a nucleic acid sequence, such as de-methylation, methylation, hypomethylation and hypermethylation. DNA methylation is an important regulator of gene transcription and a large body of evidence has demonstrated that aberrant DNA methylation is associated with unscheduled gene silencing, and the genes with high levels of 5-methylcytosine in their promoter region are transcriptionally silent. Aberrant DNA methylation patterns have been associated with a large number of human malignancies and found in two distinct forms: hypermethylation and hypomethylation compared to normal tissue. Hypermethylation is one of the major epigenetic modifications that repress transcription via promoter region of tumour suppressor genes. Hypermethylation typically occurs at CpG islands in the promoter region and is associated with gene inactivation. Global hypomethylation has also been shown to be causally related to the development and progression of cancer through different mechanisms. Other chemical modifications to the DNA that are causally related to cancer will be known to those in the art.

### Detection of Genetic Markers

Any one or combination of methods may be used for detecting the different types of genetic markers from the patient's isolated nucleic acid. Suitable methods include real-time or quantitative PCR, digital PCR, PCR in flowing or stationary droplets, well plates, slugs or fluid flowing segments, and the like, in capillary tubes, microfluidic chips, or standard thermocycler based PCR methods known to those having ordinary skill in the art. Additional detection methods can utilize binding to microarrays for subsequent fluorescent or non-fluorescent detection, barcode mass detection using a mass spectrometric methods, detection of emitted radiowaves, detection of scattered light from aligned barcodes, fluorescence detection using quantitative PCR or digital PCR methods.

Still other techniques include, for example, Northern blot, selective hybridization, cleaved amplified polymorphic sequence analysis, short tandem repeat analysis, the use of supports coated with oligonucleotide probes, amplification of the nucleic acid by RT-PCR, quantitative PCR or ligation-PCR, etc. These methods can include the use of a nucleic acid probe (for example, an oligonucleotide) that can selectively or specifically detect the target nucleic acid in the sample to detect changes at the level of a single nucleotide polymorphism, whole DNA-fingerprint analysis, allele specific analysis. Amplification is accomplished according to various methods known to the person skilled in the art, such as PCR, LCR, transcription-mediated amplification (TMA), strand-displacement amplification (SDA), NASBA, the use of allele-specific oligonucleotides (ASO), allele-specific amplification, Southern blot, single-strand conformational analysis (SSCA), in-situ hybridization (e.g., FISH), migration on a gel, heteroduplex analysis, etc. If necessary, the quantity of nucleic acid detected can be compared to a reference value, for example a median or mean value observed in patients who do not have cancer, or to a value measured in parallel in a non-cancerous sample. Thus, it is possible to demonstrate a variation in the level of expression.

Preferably, the detection of the different types of genetic markers is achieved in a single assay format. In a particular embodiment, the different types of genetic markers are detected via sequencing (e.g., single molecule sequencing).

Sequencing may be achieved by any method known in the art. DNA sequencing techniques include classic di-deoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of separated molecules has more recently been demonstrated by sequential or single extension reactions using polymerases or ligases as well as by single or sequential differential hybridizations with libraries of probes.

A sequencing technology that can be used in the methods of the invention includes, for example, Helicos True Single Molecule Sequencing (tSMS) (Harris T. D. et al. (2008) Science 320:106-109). In the tSMS technique, a DNA sample is cleaved into strands of approximately 100 to 200 nucleotides, and a polyA sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. The templates can be at a density of about 100 million templates/cm2. The flow cell is then loaded into an instrument, e.g., HeliScope™ sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are detected by imaging the flow cell surface. After imaging, a cleavage step removes the fluorescent label, and the process is repeated with other fluorescently labeled nucleotides until the desired read length is achieved. Sequence information is collected with each nucleotide addition step. Further description of tSMS is shown for example in Lapidus et al. (U.S. Pat. No. 7,169,560), Lapidus et al. (U.S. patent application number 2009/0191565), Quake et al. (U.S. Pat. No. 6,818,395), Harris (U.S. Pat. No. 7,282,337), Quake et al. (U.S. patent application number 2002/0164629), and Braslaysky, et al., PNAS (USA), 100: 3960-3964 (2003), the contents of each of these references is incorporated by reference herein in its entirety.

Another example of a sequencing technology that can be used in the methods of the invention is 454 sequencing (Roche) (Margulies, M et al. 2005, Nature, 437, 376-380). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

Another example of a sequencing technology that can be used in the methods of the invention is SOLiD technology (Applied Biosystems). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

Another example of a sequencing technology that can be used in the methods of the invention is Ion Torrent sequencing (U.S. patent application numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982), the content of each of which is incorporated by reference herein in its entirety. In Ion Torrent sequencing, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to a surface and is attached at a resolution such that the fragments are individually resolvable. Addition of one or more nucleotides releases a proton (H+), which signal detected and recorded in a sequencing instrument. The signal strength is proportional to the number of nucleotides incorporated.

Another example of a sequencing technology that can be used in the methods of the invention is Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

Another example of a sequencing technology that can be used in the methods of the invention includes the single molecule, real-time (SMRT) technology of Pacific Biosciences. In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in an out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a sequencing technology that can be used in the methods of the invention is nanopore sequencing (Soni G V and Meller A. (2007) Clin Chem 53: 1996-2001). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

Another example of a sequencing technology that can be used in the methods of the invention involves using a chemical-sensitive field effect transistor (chemFET) array to sequence DNA (for example, as described in US Patent Application Publication No. 20090026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

Another example of a sequencing technique that can be used in the methods of the invention involves using an electron microscope (Moudrianakis E. N. and Beer M. Proc Natl Acad Sci USA. 1965 March; 53:564-71). In one example of the technique, individual DNA molecules are labeled using metallic labels that are distinguishable using an electron microscope. These molecules are then stretched on a flat surface and imaged using an electron microscope to measure sequences.

Chemical modifications to a nucleic acid sequence can be detected known methods in the art. In other certain aspects, DNA methylation is detected. Methods for DNA methylation analysis can be divided roughly into two types: global and gene-specific methylation analysis. For global methylation analysis, there are methods which measure the overall level of methyl cytosines in genome such as chromatographic methods and methyl accepting capacity assay.

For gene-specific methylation analysis, a large number of techniques have been developed. Most early studies used methylation sensitive restriction enzymes to digest DNA followed by Southern detection or PCR amplification. Recently, bisulfite reaction based methods have become very popular such as methylation specific PCR (MSP), bisulfite genomic sequencing PCR. Bisulfite Modification (Conversion) uses sodium bisulfite to convert unmethylated cytosines to uracils and subsequently detects methylated cytosines using methylation specific PCR (MSP) technique or bisulfite genomic sequencing after PCR amplification with or without cloning. Bisulfite genomic sequencing allows precise analysis of methylation in a certain region by converting all nonmethylated cytosines into thymines, while methylated cytosines remain unchanged. This method requires small amount of genomic DNA and therefore seems to be very useful for the analysis of clinical samples, where the material amount is limited. A protocol has been developed for handling small numbers of cells and little/limited DNA. The protocol is based on a strategy using agarose embedded DNA. This physical trapping helps to avoid DNA loss during the various incubation steps while maintaining a good bisulphite conversion rate.

Additionally, in order to identify unknown methylation hot-spots or methylated CpG islands in the genome, several of genome-wide screen methods have been invented such as Restriction Landmark Genomic Scanning for Methylation (RLGS-M), and CpG island microarray.

### Examples of Cancer-Related Biomarkers

Methods of the invention are particularly suited for the detection and diagnosis of cancer, including, but not limited to: Acute lymphoblastic leukemia; Acute myeloid leukemia; Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liposarcoma; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Macroglobulinemia, Waldenstrom; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sézary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma—see Skin cancer (nonmelanoma); Squamous neck cancer with occult primary, metastatic; Stomach cancer; Supratentorial primitive neuroectodermal tumor, childhood; T-Cell lymphoma, cutaneous—see Mycosis Fungoides and Sézary syndrome; Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenström macroglobulinemia; Wilms tumor (kidney cancer), childhood.

As such, genes thought or known to be correlated with cancer are included as targets for the methods of the present invention. The methods of the invention are not limited to any one particular gene or DNA target sequence and may include one or more genes that encode for growth factors, apoptosis inhibitors, tumor suppressor genes or other proteins known by those in the art. Example of genes and their related homologues include, but are not limited to: v-abl Abelson murine leukemia viral oncogene homolog 1; v-abl Abelson murine leukemia viral oncogene homolog 2; acyl-CoA synthetase long-chain family member 3; AF15q14 protein; ALL1-fused gene from chromosome lq; SH3 protein interacting with Nck, 90 kDa (ALL1 fused gene from 3p21); ALL1 fused gene from 5q31; A kinase (PRKA) anchor protein (yotiao) 9; v-akt murine thymoma viral oncogene homolog 1; v-akt murine thymoma viral oncogene homolog 2; aldehyde dehydrogenase 2 family (mitochondrial); anaplastic lymphoma kinase (Ki-1); KIAA1618 protein; adenomatous polyposis of the colon gene; RHO guanine nucleotide exchange factor (GEF) 12 (LARG); RAS homolog gene family, member H (TTF); AT rich interactive domain 1A (SWI-like); AT rich interactive domain 2; aryl hydrocarbon receptor nuclear translocator; alveolar soft part sarcoma chromosome region, candidate 1; additional sex combs like 1; activating transcription factor 1; 5-aminoimidazole-4-carboxamide ribonucleotide formyltransferase/IMP cyclohydrolase; ataxia telangiectasia mutated; alpha thalassemia/mental retardation syndrome X-linked; BRCA1 associated protein-1 (ubiquitin carboxy-terminal hydrolase); B-cell CLL/lymphoma 10; B-cell CLL/lymphoma 11A; B-cell CLL/lymphoma 11B (CTIP2); B-cell CLL/lymphoma 2; B-cell CLL/lymphoma 3; B-cell CLL/lymphoma 5; B-cell CLL/lymphoma 6; B-cell CLL/lymphoma 7A; B-cell CLL/lymphoma 9; BCL6 corepressor; breakpoint cluster region; folliculin, Birt-Hogg-Dube syndrome; baculoviral IAP repeat-containing 3; Bloom Syndrome; bone morphogenetic protein receptor, type IA; v-raf murine sarcoma viral oncogene homolog B1; familial breast/ovarian cancer gene 1; familial breast/ovarian cancer gene 2; bromodomain containing 3; bromodomain containing 4; BRCA1 interacting protein C-terminal helicase 1; B-cell translocation gene 1, anti-proliferative; BUB 1 budding uninhibited by benzimidazoles 1 homolog beta (yeast); chromosome 12 open reading frame 9; chromosome 15 open reading frame 21; chromosome 15 open reading frame 55; chromosome 16 open reading frame 75; calcium activated nucleotidase 1; caspase recruitment domain family, member 11; cysteinyl-tRNA synthetase; core-binding factor, runt domain, alpha subunit 2;translocated to, 1 (ETO); core-binding factor, runt domain, alpha subunit 2 translocated to 3 (MTG-16); core-binding factor, beta subunit; Cas-Br-M (murine) ecotropic retroviral transforming; Cas-Br-M (murine) ecotropic retroviral transforming sequence b; Cas-Br-M (murine) ecotropic retroviral transforming sequence c; cyclin B1 interacting protein 1, E3 ubiquitin protein ligase; cyclin D1; cyclin D2; cyclin D3; cyclin E1; programmed cell death 1 ligand 2; CD274 molecule; CD74 molecule, major histocompatibility complex, class II invariant chain; CD79a molecule, immunoglobulin-associated alpha; CD79b molecule, immunoglobulin-associated beta; cadherin 1, type 1, E-cadherin (epithelial) (ECAD); cadherin 11, type 2, OB-cadherin (osteoblast); cyclin-dependent kinase 12; cyclin-dependent kinase 4; cyclin-dependent kinase 6; cyclin-dependent kinase inhibitor 2A (p16(INK4a)) gene; cyclin-dependent kinase inhibitor 2A-p14ARF protein; cyclin-dependent kinase inhibitor 2C (p18, inhibits CDK4); caudal type homeo box transcription factor 2; CCAAT/enhancer binding protein (C/EBP), alpha; centrosomal protein 1; coiled-coil-helix-coiled-coil-helix domain containing 7; CHK2 checkpoint homolog (S. pombe); cysteine-rich hydrophobic domain 2; chimerin (chimaerin) 1; capicua homolog; class II, major histocompatibility complex, transactivator; clathrin, heavy polypeptide (Hc); clathrin, heavy polypeptide-like 1; chemokine orphan receptor 1; collagen, type I, alpha 1; core promoter element binding protein (KLF6); cytochrome c oxidase subunit VIc; cAMP responsive element binding protein 1; cAMP responsive element binding protein 3-like 1; cAMP responsive element binding protein 3-like 2; CREB binding protein (CBP); cytokine receptor-like factor 2; CREB regulated transcription coactivator 3; catenin (cadherin-associated protein), beta 1; familial cylindromatosis gene; DNA segment on chromosome 10 (unique) 170, H4 gene (PTC1); death-domain associated protein; damage-specific DNA binding protein 2; DNA-damage-inducible transcript 3; DEAD (Asp-Glu-Ala-Asp) box polypeptide 10; DEAD (Asp-Glu-Ala-Asp) box polypeptide 5; DEAD (Asp-Glu-Ala-Asp) box polypeptide 6; DEK oncogene (DNA binding); dicer 1, ribonuclease type III ; DNA (cytosine-5-)-methyltransferase 3 alpha; double homeobox, 4; early B-cell factor 1; epidermal growth factor receptor (erythroblastic leukemia viral (v-erb-b) oncogene homolog, avian); eukaryotic translation initiation factor 4A, isoform 2; E74-like factor 4 (ets domain transcription factor); ELK4, ETS-domain protein (SRF accessory protein 1); ELKS protein; ELL gene (11-19 lysine-rich leukemia gene); elastin; echinoderm microtubule associated protein like 4; 300 kd E1A-Binding protein gene; epidermal growth factor receptor pathway substrate 15 (AF1p); v-erb-b2 erythroblastic leukemia viral oncogene homolog 2, neuro/glioblastoma derived oncogene homolog (avian); excision repair cross-complementing rodent repair deficiency, complementation group 2 (xeroderma pigmentosum D); excision repair cross-complementing rodent repair deficiency, complementation group 3 (xeroderma pigmentosum group B complementing); excision repair cross-complementing rodent repair deficiency, complementation group 4; excision repair cross-complementing rodent repair deficiency, complementation group 5 (xeroderma pigmentosum, complementation group G (Cockayne syndrome)); v-ets erythroblastosis virus E26 oncogene like (avian); ets variant gene 1; ets variant gene 4 (E1A enhancer binding protein, E1AF); ets variant gene 5; ets variant gene 6 (TEL oncogene); ecotropic viral integration site 1; Ewing sarcoma breakpoint region 1 (EWS); multiple exostoses type 1 gene; multiple exostoses type 2 gene; enhancer of zeste homolog 2; fatty-acid-coenzyme A ligase, long-chain 6; family with sequence similarity 22, member A; family with sequence similarity 22, member B; family with sequence similarity 46, member C; Fanconi anemia, complementation group A; Fanconi anemia, complementation group C; Fanconi anemia, complementation group D2; Fanconi anemia, complementation group E; Fanconi anemia, complementation group F; Fanconi anemia, complementation group G; F-box protein 11; F-box and WD-40 domain protein 7 (archipelago homolog, Drosophila); Fc fragment of IgG, low affinity IIb, receptor for (CD32); FEV protein—(HSRNAFEV); fibroblast growth factor receptor 1(FGFR1); FGFR1 oncogene partner (FOP); fibroblast growth factor receptor 3 (FGFR3); fibroblast growth factor receptor 2 (FGFR2); fumarate hydratase; fragile histidine triad gene; FIP1 like 1 (S. cerevisiae); Friend leukemia virus integration 1; BX648577, FLJ27352 hypothetical LOC145788; fms-related tyrosine kinase 3; formin binding protein 1 (FBP17); forkhead box L2; forkhead box O1A (FKHR); forkhead box O3A; forkhead box P1; follistatin-like 3 (secreted glycoprotein); far upstream element (FUSE) binding protein 1; fusion, derived from t(12;16) malignant liposarcoma; follicular lymphoma variant translocation 1; growth arrest-specific 7; GATA binding protein 1 (globin transcription factor 1); GATA binding protein 2; GATA binding protein 3; guanine monphosphate synthetase; guanine nucleotide binding protein (G protein), alpha 11 (Gq class); guanine nucleotide binding protein (G protein), q polypeptide; guanine nucleotide binding protein (G protein), alpha stimulating activity polypeptide 1; golgi autoantigen, golgin subfamily a, 5 (PTC5); golgi associated PDZ and coiled-coil motif containing; glypican 3; gephyrin (GPH); GTPase regulator associated with focal adhesion kinase pp125(FAK); sperm antigen HCMOGT-1; ATP_GTP binding protein; enhancer of invasion 10-fused to HMGA2; homocysteine-inducible, endoplasmic reticulum stress-inducible, ubiquitin-like domain member 1; hairy/enhancer-of-split related with YRPW motif 1; huntingtin interacting protein 1; histone 1, H4i (H4FM); hepatic leukemia factor; homeo box HB9; high mobility group AT-hook 1; high mobility group AT-hook 2 (HMGIC); heterogeneous nuclear ribonucleoprotein A2/B1; hook homolog 3; homeo box All; homeo box A13; homeo box A9; homeo box C11; homeo box C13; homeo box D11; homeo box D13; v-Ha-ras Harvey rat sarcoma viral oncogene homolog; hyperparathyroidism 2 ; heat shock 90 kDa protein 1, alpha; heat shock 90 kDa protein 1, beta; isocitrate dehydrogenase 1 (NADP+), soluble; socitrate dehydrogenase 2 (NADP+), mitochondrial ; immunoglobulin heavy locus; immunoglobulin kappa locus; immunoglobulin lambda locus; IKAROS family zinc finger 1; interleukin 2; interleukin 21 receptor; interleukin 6 signal transducer (gp130, oncostatin M receptor); interleukin 7 receptor; interferon regulatory factor 4; immunoglobulin superfamily receptor translocation associated 1; IL2-inducible T-cell kinase; Janus kinase 1; Janus kinase 2 ; Janus kinase 3; juxtaposed with another zinc finger gene 1; jun oncogene; lysine (K)-specific demethylase 5A, JARID1A; lysine (K)-specific demethylase 5C (JARID1C); lysine (K)-specific demethylase 6A, UTX; vascular endothelial growth factor receptor 2; KIAA1549; v-kit Hardy-Zuckerman 4 feline sarcoma viral oncogene homolog; kallikrein-related peptidase 2; v-Ki-ras2 Kirsten rat sarcoma 2 viral oncogene homolog; kinectin 1 (kinesin receptor); lymphoid nuclear protein related to AF4; LIM and SH3 protein 1; lymphocyte-specific protein tyrosine kinase; lymphocyte cytosolic protein 1 (L-plastin); leukemia-associated protein with a CXXC domain; lipoma HMGIC fusion partner; leukemia inhibitory factor receptor; LIM domain only 1 (rhombotin 1) (RBTN1); LIM domain only 2 (rhombotin-like 1) (RBTN2); LIM domain containing preferred translocation partner in lipoma; lymphoblastic leukemia derived sequence 1; Homolog of Drosophila Mothers Against Decapentaplegic 4 gene; v-maf musculoaponeurotic fibrosarcoma oncogene homolog; v-maf musculoaponeurotic fibrosarcoma oncogene homolog B (avian); mucosa associated lymphoid tissue lymphoma translocation gene 1; mastermind-like 2 (Drosophila); mitogen-activated protein kinase kinase 4; Mdm2 p53 binding protein homolog; Mdm4 p53 binding protein homolog; myelodysplasia syndrome 1; myelodysplastic syndrome 2; mucoepidermoid translocated 1; mediator complex subunit 12; multiple endocrine neoplasia type 1 gene; met proto-oncogene (hepatocyte growth factor receptor); MHC class II transactivator; microphthalmia-associated transcription factor; megakaryoblastic leukemia (translocation) 1; myeloid leukemia factor 1; E.coli MutL homolog gene; myeloid/lymphoid or mixed-lineage leukemia (trithorax homolog, Drosophila); myeloid/lymphoid or mixed-lineage leukemia 2; myeloid/lymphoid or mixed-lineage leukemia 3; myeloid/lymphoid or mixed-lineage leukemia (trithorax homolog, Drosophila); translocated to, 1 (ENL); myeloid/lymphoid or mixed-lineage leukemia (trithorax homolog, Drosophila); translocated to, 10 (AF10); myeloid/lymphoid or mixed-lineage leukemia (trithorax homolog, Drosophila); translocated to, 2 (AF4); myeloid/lymphoid or mixed-lineage leukemia (trithorax homolog, Drosophila); translocated to, 3 (AF9); myeloid/lymphoid or mixed-lineage leukemia (trithorax homolog, Drosophila); translocated to, 4 (AF6); myeloid/lymphoid or mixed-lineage leukemia (trithorax homolog, Drosophila); translocated to, 6 (AF17); myeloid/lymphoid or mixed-lineage leukemia (trithorax homolog, Drosophila); translocated to, 7 (AFX1); meningioma (disrupted in balanced translocation) 1; myeloproliferative leukemia virus oncogene, thrombopoietin receptor; MLL septin-like fusion; mutS homolog 2 (E. coli); mutS homolog 6 (E. coli); musashi homolog 2 (Drosophila); moesin; mature T-cell proliferation 1; mucin 1, transmembrane; mutY homolog (E. coli); v-myb myeloblastosis viral oncogene homolog; v-myc myelocytomatosis viral oncogene homolog (avian); v-myc myelocytomatosis viral oncogene homolog 1, lung carcinoma derived (avian); v-myc myelocytomatosis viral related oncogene, neuroblastoma derived (avian); myeloid differentiation primary response gene (88); myosin, heavy polypeptide 11, smooth muscle; myosin, heavy polypeptide 9, non-muscle; MYST histone acetyltransferase (monocytic leukemia) 4 (MORF); nascent-polypeptide-associated complex alpha polypeptide; Nijmegen breakage syndrome 1 (nibrin); nuclear receptor coactivator 1; nuclear receptor coactivator 2 (TIF2); nuclear receptor coactivator 4-PTC3 (ELE1) N-myc downstream regulated 1; neurofibromatosis type 1 gene; neurofibromatosis type 2 gene; nuclear factor (erythroid-derived 2)-like 2 (NRF2); nuclear factor I/B; nuclear factor of kappa light polypeptide gene enhancer in B-cells 2 (p49/p100); ninein (GSK3B interacting protein); NK2 homeobox 1; non-POU domain containing, octamer-binding; Notch homolog 1, translocation-associated (Drosophila) (TAN1); Notch homolog 2; nucleophosmin (nucleolar phosphoprotein B23, numatrin); nuclear receptor subfamily 4, group A, member 3 (NOR1); neuroblastoma RAS viral (v-ras) oncogene homolog; nuclear receptor binding SET domain protein 1; neurotrophic tyrosine kinase, receptor, type 1; neurotrophic tyrosine kinase, receptor, type 3; nuclear mitotic apparatus protein 1; nucleoporin 214 kDa (CAN); nucleoporin 98 kDa; nuclear protein in testis; oligodendrocyte lineage transcription factor 2 (BHLHB1); osteomodulin; purinergic receptor P2Y, G-protein coupled, 8; platelet-activating factor acetylhydrolase, isoform Ib, beta subunit 30 kDa; partner and localizer of BRCA2; paired box gene 3 ; paired box gene 5 (B-cell lineage specific activator protein); paired box gene 7; paired box gene 8; polybromo 1; pre-B-cell leukemia transcription factor 1; pericentriolar material 1 (PTC4); proprotein convertase subtilisin/kexin type 7; phosphodiesterase 4D interacting protein (myomegalin); platelet-derived growth factor beta polypeptide (simian sarcoma viral (v-sis) oncogene homolog); platelet-derived growth factor, alpha-receptor; platelet-derived growth factor receptor, beta polypeptide; period homolog 1 (Drosophila); paired-like homeobox 2b; phosphatidylinositol binding clathrin assembly protein (CALM); phosphoinositide-3-kinase, catalytic, alpha polypeptide; phosphoinositide-3-kinase, regulatory subunit 1 (alpha); pim-1 oncogene; pleiomorphic adenoma gene 1; promyelocytic leukemia; PMS 1 postmeiotic segregation increased 1 (S. cerevisiae); PMS2 postmeiotic segregation increased 2 (S. cerevisiae); paired mesoderm homeo box 1; peanut-like 1 (Drosophila); POU domain, class 2, associating factor 1 (OBF1); POU domain, class 5, transcription factor 1; peroxisome proliferative activated receptor, gamma; protein phosphatase 2, regulatory subunit A, alpha; papillary renal cell carcinoma (translocation-associated); PR domain containing 1, with ZNF domain; PR domain containing 16; perforin 1 (pore forming protein); protein kinase, cAMP-dependent, regulatory, type I, alpha (tissue specific extinguisher 1); PRO1073 protein (ALPHA) ; PC4 and SFRS1 interacting protein 2 (LEDGF); Homolog of Drosophila Patched gene; phosphatase and tensin homolog gene; protein tyrosine phosphatase, non-receptor type 11; rabaptin, RAB GTPase binding effector protein 1 (RABPT5); RAD51-like 1 (S. cerevisiae) (RAD51B); v-raf-1 murine leukemia viral oncogene homolog 1; ral guanine nucleotide dissociation stimulator; RAN binding protein 17; RAP1, GTP-GDP dissociation stimulator 1; retinoic acid receptor, alpha; retinoblastoma gene; RNA binding motif protein 15; RecQ protein-like 4; v-rel reticuloendotheliosis viral oncogene homolog (avian); ret proto-oncogene; v-ros UR2 sarcoma virus oncogene homolog 1 (avian); ribosomal protein L22 (EAP); ribophorin I; RUN domain containing 2A; runt-related transcription factor 1 (AML1); runt-related transcription factor binding protein 2 (MOZ/ZNF220); Shwachman-Bodian-Diamond syndrome protein; chromosome 11 open reading frame 79; succinate dehydrogenase complex, subunit B, iron sulfur (Ip); succinate dehydrogenase complex, subunit C, integral membrane protein, 15 kDa; succinate dehydrogenase complex, subunit D, integral membrane protein; septin 6; SET translocation; SET domain containing 2; splicing factor 3b, subunit 1, 155 kDa; splicing factor proline/glutamine rich(polypyrimidine tract binding protein associated); splicing factor, arginine/serine-rich 3; SH3-domain GRB2-like 1 (EEN); TALI (SCL) interrupting locus; solute carrier family 45, member 3; SWI/SNF related, matrix associated, actin dependent regulator of chromatin, subfamily a, member 4; SWI/SNF related, matrix associated, actin dependent regulator of chromatin, subfamily b, member 1; smoothened homolog (Drosophila); suppressor of cytokine signaling 1; SRY (sex determining region Y)-box 2; SLIT-ROBO Rho GTPase activating protein 3; serine/arginine-rich splicing factor 2; synovial sarcoma translocation, chromosome 18; synovial sarcoma translocation gene on chromosome 18-like 1; spectrin SH3 domain binding protein 1; synovial sarcoma, X breakpoint 1; synovial sarcoma, X breakpoint 2; synovial sarcoma, X breakpoint 4; serine/threonine kinase 11 gene (LKB1); Six-twelve leukemia gene; suppressor of fused homolog (Drosophila); suppressor of zeste 12 homolog (Drosophila); spleen tyrosine kinase; TAF15 RNA polymerase II, TATA box binding protein (TBP)-associated factor, 68 kDa; T-cell acute lymphocytic leukemia 1 (SCL); T-cell acute lymphocytic leukemia 2; transcription elongation factor A (SII), 1; transcription factor 1, hepatic (HNF1); transcription factor 12 (HTF4, helix-loop-helix transcription factors 4); transcription factor 3 (E2A immunoglobulin enhancer binding factors E12/E47); transcription factor 7-like 2; T-cell leukemia/lymphoma 1A; T-cell leukemia/lymphoma 6; tet oncogene family member 2; transcription factor binding to IGHM enhancer 3; transcription factor EB; TRK-fused gene; TCF3 (E2A) fusion partner (in childhood Leukemia); transferrin receptor (p90, CD71); thyroid hormone receptor associated protein 3 (TRAP150); transcriptional intermediary factor 1 (PTC6,TIF1A); T-cell leukemia, homeobox 1 (HOX11); T-cell leukemia, homeobox 3 (HOX11L2); transmembrane protease, serine 2; tumor necrosis factor, alpha-induced protein 3; tumor necrosis factor receptor superfamily, member 14 (herpesvirus entry mediator); tumor necrosis factor receptor superfamily, member 17; tumor necrosis factor receptor superfamily, member 6 (FAS); topoisomerase (DNA) I; tumor protein p53; tropomyosin 3; tropomyosin 4; translocated promoter region; T cell receptor alpha locus; T cell receptor beta locus; T cell receptor delta locus; tripartite motif-containing 27; tripartite motif-containing 33 (PTC7,TIF1G); thyroid hormone receptor interactor 11; tuberous sclerosis 1 gene; tuberous sclerosis 2 gene; thyroid stimulating hormone receptor; tubulin tyrosine ligase; U2 small nuclear RNA auxiliary factor 1; ubiquitin specific peptidase 6 (Tre-2 oncogene); von Hippel-Lindau syndrome gene; vesicle transport through interaction with t-SNAREs homolog 1A; Wiskott-Aldrich syndrome; Wolf-Hirschhorn syndrome candidate 1(MMSET); Wolf-Hirschhorn syndrome candidate 1-like 1 (NSD3) WNT inhibitory factor 1; Werner syndrome (RECQL2); Wilms tumour 1 gene; family with sequence similarity 123B (FAM123B); xeroderma pigmentosum, complementation group A; xeroderma pigmentosum, complementation group C; exportin 1 (CRM1 homolog, yeast); tyrosine 3-monooxygenase/tryptophan 5-monooxygenase activation protein, epsilon polypeptide (14-3-3 epsilon); zinc finger protein 145 (PLZF); zinc finger protein 198; zinc finger protein 278 (ZSG); zinc finger protein 331; zinc finger protein 384 (CIZ/NMP4); zinc finger protein 521; zinc finger protein 9 (a cellular retroviral nucleic acid binding protein); zinc finger (CCCH type), RNA-binding motif and serine/arginine rich 2.

### INCORPORATION BY REFERENCE

References and citations to other documents, such as patents, patent applications, patent publications, journals, books, web contents, have been made throughout this disclosure. All documents are hereby incorporated herein by reference in their entirety for all purposes.

### EQUIVALENTS

The invention may be embodies in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced from within.

What is claimed is:

1. A method for detection a clinical condition in a subject, said method comprising the steps of:
isolating at least one nucleic acid from a biological sample obtained from the subject;
detecting at least one sequence mutation and a chromosomal abnormality in the at least one nucleic acid in a single assay format; and
identifying a clinical condition in said subject when both the sequence mutation and the chromosomal abnormality are present.

2. The method of claim 1, wherein the at least one nucleic acid is DNA.

3. The method of claim 2, wherein the DNA is genomic DNA.

4. The method of claim 1, wherein the sequence mutation and chromosomal abnormality occur on the same chromosome.

5. The method of claim 1, wherein the sequence mutation and the chromosomal abnormality occur on different chromosomes.

6. The method of claim 1, wherein the sequence mutation is a point mutation.

7. The method of claim 1, wherein the chromosomal abnormality is loss of heterozygosity.

8. The method of claim 1, wherein the clinical condition is cancer.

9. The method of claim 8, wherein the cancer is bladder cancer.

10. The method of claim 1, wherein the detecting step further comprises detecting a chemical modification to the nucleic acid.

11. The method of claim 10, wherein the chemical modification to the nucleic acid comprises hypermethylation.

12. The method of claim 1, wherein the single assay format comprises a nucleic acid sequencing technique.

13. The method of claim 12, wherein the sequencing technique is a single molecule sequencing technique.

14. The method of claim 1, wherein a sequence mutation in FGFR3 is detected.

15. The method of claim 1, wherein a chromosomal abnormality in p53 is detected.

16. The method of claim 15, wherein the chromosomal abnormality is loss of heterzygosity.
